(19) United States
(12) Patent Application Publication
(10) Pub. No.: US 2011/0021899 A1
(43) Pub. Date: Jan. 27, 2011

(54) CONDUCTIVE POLYMER COATINGS

(75) Inventors: James H. Arps, Chanhassen, MN (US); Peter J. Barnett, Eden Prairie, MN (US); Jeffrey J. Missling, Eden Prairie, MN (US)

Correspondence Address: Pauly, Devries Smith & Deffner, L.L.C., Plaza VII, 45 South Seventh Street, Suite 3000, Minneapolis, MN, 55402-1630, US

(73) Assignee: SurModics, Inc., Eden Prairie, MN (US)

(21) Appl. No.: 12/831,539

(22) Filed: Jul. 7, 2010

Related U.S. Application Data
(60) Provisional application No. 61/227,843, filed on Jul. 23, 2009.

(51) Int. Cl. A61B 5/0408; H01B 1/16; H01B 1/18; A61K 47/02; A61K 47/04; A61K 47/34; A61K 47/32; A61N 1/04

(52) U.S. Cl. 600/372; 252/500; 252/511; 514/772.3; 514/769; 514/772.7; 514/788.1; 607/115

(57) ABSTRACT

An electrically conductive coating composition that includes a polymeric mixture, an electrically conductive material dispersed within the polymeric mixture and, optionally, one or more bioactive agents is described.

This application claims the benefit of U.S. Provisional Application No. 61/227,843, filed Jul. 23, 2009, the content of which is herein incorporated by reference in its entirety.

### TECHNICAL FIELD

In general, the invention described herein provides a coating composition for a medical device. In particular, the invention described herein provides an electrically conductive coating composition for an implantable medical device.

### BACKGROUND

Surface coatings are often used to modify the properties of medical devices and implants including, for example, surface wettability, electrical conductivity, radio-opacity, echogram visibility, coefficient of friction, etc. Coatings can also be used for the delivery of bioactive agents.

In general, the mechanical properties of polymers (flexibility, toughness, malleability, elasticity, etc.) make polymers suitable for use as coatings for implantable medical devices. However, many polymer coatings are non-conductive and function as electrical insulators.

Conductive polymers are known and include organic polymers that are capable of conducting electricity. Well-studied classes of organic conductive polymers include poly(acetylene)s, poly(pyrrole)s, poly(thiophene)s, poly(aniline)s, poly(p-phenylene sulfide), and poly(para-phenylene vinylene)s (PPV). Other less well studied conductive polymers include polyindole, polypyrene, polycarbazole, polyazulene, polyazepine, poly(fluorene)s, and polynaphthalene. However, many electrically conductive polymers have poor mechanical properties such as insolubility, intractability, poor elasticity, low resistance to water or heat, poor processibility, or in some cases, low molecular weights, which can render them unsuitable for use as coatings.

### SUMMARY

Described herein is an electrically conductive coating composition. In one embodiment, the electrically conductive coating composition includes a polymeric mixture, an electrically conductive material and a bioactive agent. In one embodiment, the electrically conductive material includes a conductive polymeric material. In another embodiment, the electrically conductive material includes biocompatible inorganic electrically conducting filler. In one embodiment, the coating composition further includes one or more bioactive agents. Also described herein is a medical device with one or more electrically conductive elements coated with the conductive coating composition and methods for coating a medical device with the coating composition.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

FIGS. 1A-F are schematic representations for various monomeric subunits for conductive polymers.

FIG. 2 is a schematic representation of a coating composition described herein.

FIG. 3 is a schematic representation of a medical device with electrically conductive elements coated with the composition described herein.

While the invention is susceptible to various modifications and alternative forms, specifics thereof have been shown by way of example and drawings, and will be described in detail. It should be understood, however, that the invention is not limited to the particular embodiments described. On the contrary, the intention is to second modifications, equivalents, and alternatives falling within the spirit and scope of the invention.

### DETAILED DESCRIPTION

Described herein is a conductive coating composition and related method for coating a medical device that includes one or more electrically conductive elements. In one embodiment, one or more electrically conductive elements include an electrode. As used herein, the term “conductive coating composition” refers to a composition that is capable of conducting sufficient electrical current for the underlying or associated device to properly function. The resistance of a coating composition is a measure of its opposition to the passage of electric current. In general, an object of uniform cross section will have a resistance proportional to its length and inversely proportional to its cross-sectional area, and proportional to the resistivity of the material. The resistivity of a conductive coating composition can vary over several orders of magnitude depending upon factors such as the materials contained within the coating composition and the thickness of the coating. For example, titanium nitride coatings are used as electrode coatings and have a resitivity in the range 3E8 ohm*cm, while another common electrode material platinum-iridium has a resistivity of 3E-5 ohm*cm. In general, a coating composition with a higher resistivity can be applied as a relatively thin coating and still allow the underlying device to properly function. Whereas, a coating composition with a lower resistivity can be applied as a relatively thick coating and still allow the underlying device to properly function. The reciprocal quantity to electrical resistivity is electrical conductivity.

Resistance to an alternating current can also be defined by the electrode impedance. For implantable devices, it is generally desirable to reduce the impedance at the interface between the electrode and the patient's tissue. One way in which impedance can be reduced is by increasing the surface area of the device or using a coating that increases the functional surface area of the device. Depending on the configuration, impedance values for implantable electrodes can range from 100 ohms to more than 1E6 ohms.

In one embodiment, the coating composition is a durable coating composition. As used herein, “durable coating composition” refers to a composition that is designed to remain on the medical device for the duration that the medical device is in use, i.e., a durable coating is not designed to degrade in vivo. In general, durable drug delivery coatings release bioactive agent by diffusion via concentration, electrical potential, and/or pressure gradients. In contrast, a “degradable coating composition” includes polymers that are broken down in vivo into biologically acceptable molecules that can be metabolized and removed from the body via normal metabolic pathways. In some instances, bioactive agent is released from a degradable drug delivery coating due to the degradation of the polymer matrix.

The electrically conductive coating composition described herein includes a non-conductive polymeric mixture and an electrically conductive material. In one embodiment, the polymer mixture includes hydrophobic polymers.

In general, polymeric mixtures suitable for use as a drug delivery matrix include a nontoxic and/or nonimmunogenic material that can control the rate at which one or more bioactive agents are released from the matrix. In one embodiment, the polymeric matrix includes one or more materials that can be varied to alter the rate at which one or more bioactive agents are released from the matrix. In one embodiment, the rate at which one or more bioactive agents are released from the polymeric matrix can be facilitated by the electrical current. In one embodiment, the bioactive agent is ionic (i.e., has a net positive or negative charge) and release is facilitated based on the basic electrical principal that oppositely charged ions attract and similarly charge ions repel. Thus, an ionized bioactive agent can be driven into a patient's tissue by electrorepulsion at the anode (for a positively charged bioactive agent) or at the cathode (for a negatively charged bioactive agent). In another embodiment, the bioactive agent neutral (i.e., does not have a net positive or negative charge). In some embodiments, the polymeric mixture also has additional mechanical properties, including, but not limited to, elasticity (e.g., a polymeric material that can be distorted through the application of force, and when the force is removed, the material returns to its original shape), for example, to allow for expansion of the underlying device or electrical conductivity, to allow for the transmission of an electrical current through the polymeric matrix, for example, for application to an electrode. Another consideration when selecting a polymeric matrix may include processability of the polymer matrix. For example, it is generally desirable to have a polymeric composition that can be readily deposited or applied to a surface or device without becoming damaged, which can result in a reduction in one or more desirable properties.

The conductive coating composition described herein has many beneficial physical, mechanical and chemical properties that can help improve the performance of an electrically conductive element of a medical device. A first desirable property of the conductive coating composition described herein is the biocompatibility of the coating, e.g., the coating results in no significant induction of inflammation or irritation when implanted. Therefore, in one embodiment, the conductive coating composition can be applied to an electrically conductive element of a medical device to improve biocompatibility of the device.

In another embodiment, the conductive polymer coating can be applied to increase the surface roughness of the underlying electrically conductive element. While not wishing to be bound by theory, it is believed that increasing the surface roughness of the device can improve soft tissue integration and permit fibrous tissue ingrowth, which can improve long term fixation and anchoring of the device. A properly anchored device will tend to have reduced mechanical movement, which can reduce the growth of connective tissue around the device surface. As such, the coating composition can result in a reduced connective tissue “capsule” forming between the device and the tissue of the patient. This can be quite significant, as the connective tissue capsule is generally not excitable, and acts as an effective extension of the distance between the device and the tissue, which can adversely affect both stimulation and sensing functions of the medical device. In yet another embodiment, the conductive coating composition can be applied to an electrically conductive element, such as an electrode, to reduce electrical impedance between the electrically conductive element and the patient's tissue. In another embodiment, the conductive polymer coating can be applied to increase the surface area of the underlying electrically conductive element. The high surface area and reduced impedance of the conductive polymeric coating may result in decreased voltage demands for the underlying device, which can reduce demands on the battery and, potentially, reduce the size of the battery pack itself.

Additionally, in other embodiments, the conductive coating composition can be applied to an electrically conductive element, such as an electrode, to deliver one or more bioactive agents to the tissues of the patient. When in use, the conductive coating composition is in intimate contact with the patient's tissue, thus facilitating drug transfer to desired location. In many instances, it may be desirable to have localized drug delivery associated with an implanted medical device. For example, steroids are commonly applied to electrodes in a solid drug form prior to implantation. However, most of the applied steroids dissolve too quickly in vivo to effectively reduce the inflammatory reaction associated with implantation. In contrast, the conductive coating composition described herein provides a matrix for the controlled release of bioactive agents, such as steroids, and since electrical current is able to be transmitted through the conductive coating composition, masking processes are not necessary.

In addition to the properties described above, the conductive coating composition described herein has excellent mechanical integrity. In contrast, many known conductive polymers are relatively brittle and their adhesion as coatings to metal substrates is relatively unproven. Furthermore, known conductive polymers are typically applied to a surface by electrochemical deposition. However, the consistency of coatings deposited by electrochemical deposition can be difficult to control as the coating bath chemistry can change over time and the solution resistance can change as the coating is built up and the bath is depleted. In contrast, the conductive coating composition described herein can be applied using a variety of coating methods, including, but not limited to spray coating methods such as ultrasonic or pneumatic spray coating, or dipping processes, which generally provide more consistent coatings than electrochemical deposition processes.

In general, the polymeric mixture provides a coating composition that is generally hydrophobic, such that the coating composition is generally limited in the intake of aqueous fluids. For example, many embodiments are coating compositions including two or more hydrophobic polymers wherein the resulting coating shows <10% (wt) weight change when exposed to water, and in some embodiments <5% (wt) weight change when exposed to water.

### Polymeric Mixture

In one embodiment, the conductive coating composition includes polymers or a polymeric mixture. The polymers in the polymeric mixture can be natural or synthetic polymers. In one embodiment, the coating includes a mixture of hydrophobic polymers.

In one embodiment, the polymeric mixture includes one or more natural polymers. In one embodiment, the polymeric mixture includes a naturally occurring phospholipid polymer such as a phosphorylcholine-based polymer. In general, phosphorylcholine is a phospholipid polymer that is believed to improve surface biocompatibility and may lower the risk of inflammation or thrombosis. Furthermore, phosphorycholine polymers can be used for local delivery of one or more bioactive agents. In another embodiment, the polymeric mixture includes one or more synthetic phospholipid polymers. In one embodiment, the polymeric matrix includes a methacrylate-based copolymer that includes a synthetic form of phosphorylcholine, hereinafter referred to as “a phosphorylcholine (PC)-based polymer.” Synthetic phosphorylcholine coatings are known. See, for example, US Published Application No. 2008/0292778, the disclosure of which is hereby incorporated by reference.

In another embodiment, the polymeric mixture includes one or more block copolymers, such as block polymers having polyalkylene blocks and poly(vinyl aromatic) blocks, including, but not limited to block copolymers containing polyisobutylene and polystyrene blocks, for example, polystyrene-polyisobutylene-polystyrene triblock copolymers (SIBS copolymers), described in U.S. Pat. No. 6,545,097 to Pinchuk et al., which is hereby incorporated by reference in its entirety. These copolymers have proven to be valuable elastomers for use implantable or insertable medical device applications due to their excellent strength, biocompatibility and biostability.

In another embodiment, the polymeric mixture includes one or more fully or partially fluorinated polymers, including, but not limited to poly(tetrafluoro ethylene) (PTFE), expanded poly(tetrafluoro ethylene) (ePTFE), poly(vinylidene fluoride) (PVDF), and poly(vinylidene fluoride-co-hexafluoropropene) (PVDF-HFP), which can be impregnated with one or more bioactive agents for localized drug delivery.

In yet another embodiment, the polymer mixture includes a first polymer component and a second polymer component. In one embodiment the mixture includes one or more polymers selected from poly(alkyl)(meth)acrylates and poly(aromatic(meth)acrylates), or combinations and mixtures thereof as a first polymeric component, where “(meth)” includes such molecules in either the acrylic and/or methacrylic form (corresponding to the acrylates and/or methacrylates, respectively). In one embodiment, the composition includes one or more polymers selected from: (i) ethylene copolymers with other alkylenes, (ii) polybutenes, (iii) aromatic group-containing copolymers, (iv) epichlorohydrin-containing polymers, (v) poly(alkylene-co-alkyl(meth)acrylates), (vi) diolefin-derived, non-aromatic polymers and copolymers; (vii) and poly(ethylene-co-vinyl acetate) (“pEVA”), and mixtures and combinations thereof as a second polymeric component. Suitable polymer mixtures are described in the following commonly assigned U.S. Patents and Applications: U.S. Pat. No. 6,214,901; U.S. Pat. No. 6,344,035; U.S. Pat. No. 6,890,583; U.S. Pat. No. 7,008,667; U.S. Pat. No. 7,442,402; U.S. Pat. No. 7,541,048; US 2002/0188037; US 2005/0220839; US 2004/0220841; US 2005/0220842; US 2005/0220843; US 2005/0244459; 2005/2060246; US 2005/0220840; and US 2006/0083772, the disclosures of which are hereby incorporated by reference herein in their entirety. Many suitable polymers are commercially available from sources such as Sigma-Aldrich.

The polymeric mixture is generally useful throughout a broad spectrum of both absolute concentrations and relative concentrations of first and second polymer components. This means that the physical characteristics of the coating, such as tenacity, durability, flexibility and expandability, will typically be adequate over a broad range of polymer concentrations. In turn, the ability of the coating to control the release rates of a variety of bioactive agents can be manipulated by varying the absolute and relative concentrations of the polymers.

Examples of poly(alkyl)(meth)acrylates include those with alkyl chain lengths from at least 2 carbons and up to 8 carbons, and with molecular weights from at least about 50 kilodaltons, or at least about 100 kilodaltons, or at least about 150 kilodaltons, or at least about 200 kilodaltons, and up to about 400 kilodaltons, or up to about 500 kilodaltons, or up to about 900 kilodaltons. Unless otherwise indicated, the term “molecular weight” and all polymeric molecular weights described herein are “weight average” molecular weights (“MW”). One example of a suitable poly(alkyl)(meth)acrylate is poly n-butylmethacrylate. Such polymers are available commercially with varying inherent viscosity, solubility, and form (e.g., as crystals or powder).

In one embodiment, the poly(alkyl)methacrylate is an ester of a methacrylic acid. In one embodiment, the poly(alkyl)methacrylate includes poly(n-butyl methacrylate). Examples of other polymers include, but are not limited to, poly(n-butyl methacrylate-co-methyl methacrylate), with a monomer ratio of 3:1, poly(n-butyl methacrylate-co-isobutyl methacrylate), with a monomer ratio of 1:1 and poly(t-butyl methacrylate). Such polymers are available commercially (e.g., from Sigma-Aldrich, Milwaukee, Wis.) with molecular weights ranging from at least about 150 kilodaltons and up to about 350 kilodaltons, and with varying inherent viscosities, solubilities and forms (e.g., as slabs, granules, beads, crystals or powder).

Examples of suitable poly(aromatic(meth)acrylates) include poly(aryl(meth)acrylates), poly(aralkyl(meth)acrylates), poly(alkaryl(meth)acrylates), poly(aryloxyalkyl(meth)acrylates), and poly(alkoxyaryl(meth)acrylates). Such terms are used to describe polymeric structures wherein at least one carbon chain and at least one aromatic ring are combined with (meth)acrylic groups, typically esters, to provide a composition. For instance, and more specifically, a poly(aralkyl(meth)acrylate) can be made from aromatic esters derived from alcohols also containing aromatic moieties, such as benzyl alcohol. Similarly, a poly(alkaryl(meth)acrylate) can be made from aromatic esters derived from aromatic alcohols such as p-anisole. Suitable poly(aromatic(meth)acrylates) include aryl groups having from 6 to 16 carbon atoms and with molecular weights from about 50 to about 900 kilodaltons. Examples of suitable poly(aryl(meth)acrylates) include poly(9-anthracenyl methacrylate), poly(chlorophenyl acrylate), poly(methacryloxy-2-hydroxybenzophenone), poly(methacryloxybenzotriazole), poly(naphthyl acrylate), poly(naphthylmethacrylate), poly-4-nitrophenylacrylate, poly(pentachloro(bromo, fluoro)acrylate) and methacrylate, poly(phenyl acrylate) and poly(phenyl methacrylate). Examples of suitable poly(aralkyl(meth)acrylates) include poly(benzyl acrylate), poly(benzyl methacrylate), poly(2-phenethyl acrylate), poly(2-phenethyl methacrylate) and poly(1-pyrenylmethyl methacrylate). Examples of suitable poly(alkaryl(meth)acrylates include poly(4-sec-butylphenyl methacrylate), poly(3-ethylphenyl acrylate), and poly(2-methyl-1-naphthyl methacrylate). Examples of suitable poly(aryloxyalkyl(meth)acrylates) include poly(phenoxyethyl acrylate), poly(phenoxyethyl methacrylate), and poly(polyethylene glycol phenyl ether acrylate) and poly(polyethylene glycol phenyl ether methacrylate) with varying polyethylene glycol molecular weights. Examples of suitable poly(alkoxyaryl(meth)acrylates) include poly(4-methoxyphenyl methacrylate), poly(2-ethoxyphenyl acrylate) and poly(2-methoxynaphthyl acrylate).

Acrylate or methacrylate monomers or polymers and/or their parent alcohols are commercially available from Sigma-Aldrich (Milwaukee, Wis.) or from Polysciences, Inc, (Warrington, Pa.).

Ethylene copolymers with other alkylenes can include straight chain and branched alkylenes, as well as substituted or unsubstituted alkylenes. Examples include copolymers prepared from alkylenes include copolymers having at least 3 branched or linear carbon atoms up to about 8 branched or linear carbon atoms. In one embodiment, the alkylene copolymers include alkylene groups having at least 3 branched or linear carbon atoms up to about to 4 branched or linear carbon atoms. In one embodiment, the alkylene group contains 3 carbon atoms (e.g., propylene). In some embodiments, the alkylene is a straight chain alkylene (e.g., 1-alkylene).

In one embodiment, the ethylene copolymer has at least about 20% ethylene (based on moles). In another embodiment, the ethylene copolymer has at least about 35% (mole) of ethylene. In one embodiment, the ethylene copolymer has up to about 80% (mole) of ethylene. In another embodiment, the ethylene copolymer has up to about 90% (mole) of ethylene. Such copolymers will generally have a molecular weight of at least about 30 kilodaltons and up to about 500 kilodaltons. Examples of such copolymers include poly(ethylene-co-propylene), poly(ethylene-co-1-butene), polyethylene-co-1-butene-co-1-hexene) and/or poly(ethylene-co-1-octene).

Examples of particular copolymers include poly(ethylene-co-propylene) random copolymers in which the copolymer contains at least about 35% (mole) ethylene, or at least about 55% (mole) of ethylene. In another embodiment, the copolymer includes up to about 65% (mole) of ethylene. In general, the molecular weight of the copolymer is at least about 50 kilodaltons, or at least about 100 kilodaltons. In another embodiment, the molecular weight is up to about 200 kilodaltons, or up to about 250 kilodaltons.

Copolymers can be provided in the form of random terpolymers prepared by the polymerization of both ethylene and propylene with, optionally, one or more additional diene monomers, including, but not limited to, ethylidene norborane, dicyclopentadiene and/or hexadiene. Various terpolymers of this type can include up to about 5% (mole) of the third diene monomer.

Other examples of suitable copolymers are commercially available from sources such as Sigma-Aldrich. The copolymers and their related descriptions may be found in the 2003-2004 Aldrich Handbook of Fine Chemicals and Laboratory Equipment, the entire contents of which are incorporated by reference herein. Examples of such copolymers include, but are not limited to poly(ethylene-co-propylene), poly(ethylene-co-1-butene), poly(ethylene-co-1-butene-co-1-hexene), poly(ethylene-co-1-octene) and poly(ethylene-co-propylene-co-5-methylene-2-norborene).

“Polybutenes” include polymers derived by homopolymerizing or randomly interpolymerizing isobutylene, 1-butene and/or 2-butene. The polybutene can be a homopolymer of any of the isomers or it can be a copolymer or a terpolymer of any of the monomers in any ratio. In various embodiments, the polybutene contains at least about 90% (wt) of isobutylene or 1-butene, and in some embodiments, the polybutene contains at least about 90% (wt) of isobutylene. The polybutene may contain non-interfering amounts of other ingredients or additives, for instance it can contain up to 1000 ppm of an antioxidant (e.g., 2,6-di-tert-butyl-methylphenol).

In one embodiment, the polybutene has a molecular weight of at least about 100 kilodaltons, or at least about 150 kilodaltons, or at least about 200 kilodaltons, or at least about 350 kilodaltons. In one embodiment, the polybutene has a molecular weight of up to about 250 kilodaltons, or up to about 500 kilodaltons, or up to about 600 kilodaltons, or up to about 1,000 kilodaltons. Polybutenes having a molecular weight greater than about 600 kilodaltons, including greater than 1,000 kilodaltons are available but are expected to be more difficult to work with. Other examples of suitable copolymers of this type are commercially available from sources such as Sigma-Aldrich.

Aromatic group-containing copolymers, include random copolymers, block copolymers and graft copolymers. In some embodiments, the aromatic group is incorporated into the copolymer via the polymerization of styrene, and in other embodiments, the random copolymer is a copolymer derived from copolymerization of styrene monomer and one or more monomers selected from butadiene, isoprene, acrylonitrile, a C₁-C₄ alkyl(meth)acrylate (e.g., methyl methacrylate) and/or butene (e.g., isobutylene). Useful block copolymers include copolymer containing (a) blocks of polystyrene, (b) blocks of a polyolefin selected from polybutadiene, polyisoprene and/or polybutene (e.g., polyisobutylene), and (c) optionally a third monomer (e.g., ethylene) copolymerized in the polyolefin block.

In one embodiment, the aromatic group-containing copolymers contains at least about 10% (wt) and up to about 50% (wt) of polymerized aromatic monomer. In one embodiment, the molecular weight of the copolymer is at least about 50 kilodaltons, or at least about 100 kilodaltons, or at least about 300 kilodaltons. In one embodiment, the molecular weight of the copolymer is up to about 300 kilodaltons, or up to about 500 kilodaltons.

Other examples of suitable copolymers include, but are not limited to, poly(styrene-co-butadiene) (random), polystyrene-block-polybutadiene, polystyrene-block-polybutadiene-block-polystyrene, polystyrene-block-poly(ethylene-ran-butylene)-block-polystyrene, polystyrene-block-polyisoprene-block-polystyrene, polystyrene-block-polyisobutylene-block-polystyrene, poly(styrene-co-acrylonitrile), poly(styrene-co-butadiene-co-acrylonitrile) and poly(styrene-co-butadiene-co-methyl methacrylate).

In one embodiment, epichlorohydrin homopolymers and poly(epichlorohydrin-co-alkylene oxide)copolymers can include ethylene oxide as the copolymerized alkylene oxide. In one embodiments the epichlorohydrin content of the epichlorohydrin-containing polymer is at least about 30% (wt), or at least about 50% (wt) and up to about 100% (wt). In some embodiments, the epichlorohydrin-containing polymers have a MW of at least about 100 kilodaltons. In one embodiment, the epichlorohydrin-containing polymers have a MW of up to about 300 kilodaltons.

Other examples of suitable copolymers of this type are commercially available from sources such as Sigma-Aldrich and include, but are not limited to, polyepichlorohydrin and poly(epichlorohydrin-co-ethylene oxide).

Poly(alkylene-co-alkyl(meth)acrylates) include those copolymers in which the alkyl groups are either linear or branched, and substituted or unsubstituted with non-interfering groups or atoms. In one embodiment, alkyl groups include at least 1 carbon atom and up to 4 carbon atoms, or up to 8 carbon atoms, inclusive. In one example, the alkyl group is methyl.

In one embodiment, copolymers include such alkyl groups with at least about 15% (wt) alkyl acrylate and up to about 80% (wt) of alkyl acrylate. When the alkyl group is methyl, the polymer may contain at least about 20% (wt), or at least about 25% (wt) and up to about 30% (wt) or at least about 40% (wt) methyl acrylate. When the alkyl group is ethyl, the polymer can include at least about 15% (wt) and up to about 40% ethyl acrylate. When the alkyl group is butyl, the polymer, can include at least about 20% and up to about 40% butyl acrylate.

The alkylene groups can include ethylene and/or propylene, and in one embodiment, the alkylene group is ethylene. In other embodiments, the (meth)acrylate includes an acrylate (i.e., no methyl substitution on the acrylate group). Various copolymers provide a molecular weight (MW) of at least about 50 kilodaltons and up to about 200 kilodaltons, or up to about 500 kilodaltons.

The glass transition temperature for these copolymers can vary depending upon the ethylene content, alkyl length on the (meth)acrylate and whether the copolymer is an acrylate or methacrylate. At higher ethylene content, the glass transition temperature tends to be lower, and closer to that of pure polyethylene (−120° C.). A longer alkyl chain also lowers the glass transition temperature. A methyl acrylate homopolymer has a glass transition temperature of about 10° C. while a butyl acrylate homopolymer has one of about −54° C.

Copolymers such as poly(ethylene-co-methyl acrylate), poly(ethylene-co-butyl acrylate) and poly(ethylene-co-2-ethylhexyl acrylate)copolymers are available commercially from sources such as Atofina Chemicals, Inc., Philadelphia, Pa., and can be prepared using methods available to those skilled in the art.

Other examples of suitable polymers are commercially available from sources such as Sigma-Aldrich and include, but are not limited to, poly(ethylene-co-methyl acrylate), poly(ethylene-co-ethyl acrylate), and poly(ethylene-co-butyl acrylate).

Diolefin-derived, non-aromatic polymers and copolymers, can include those in which the diolefin monomer used to prepare the polymer or copolymer is selected from butadiene (CH₂═CH—CH═CH₂) and/or isoprene (CH₂═CH—C(CH₃)═CH₂). A butadiene polymer can include one or more butadiene monomer units which can be selected from the monomeric unit structures (a), (b), or (c): An isoprene polymer can include one or more isoprene monomer units which can be selected from the monomeric unit structures (d), (e), (f) or (g):

In one embodiment, the polymer is a homopolymer derived from diolefin monomers or is a copolymer of diolefin monomer with non-aromatic mono-olefin monomer, and optionally, the homopolymer or copolymer can be partially hydrogenated. Such polymers can include polybutadienes containing polymerized cis-, trans- and/or 1,2-monomer units, and in some embodiments, a mixture of all three co-polymerized monomer units, and polyisoprenes containing polymerized cis-1,4- and/or trans-1,4-monomer units, polymerized 1,2-vinyl monomer units, polymerized 3,4-vinyl monomer units and/or others as described in the Encyclopedia of Chemical Technology, Vol. 8, page 915 (1993), the entire contents of which is hereby incorporated by reference.

Non-aromatic mono-olefin co-monomers include acrylonitrile, an alkyl(meth)acrylate and/or isobutylene. In on embodiment, when the mono-olefin monomer is acrylonitrile, the interpolymerized acrylonitrile is present at up to about 50% by weight; and when the mono-olefin monomer is isobutylene, the diolefin monomer is isoprene (e.g., to form what is commercially known as a “butyl rubber”). In one embodiment, the polymers and copolymers have a MW of at least about 50 kilodaltons, or at least about 100 kilodaltons, or at least about 150 kilodaltons, or at least about 200 kilodaltons. In one embodiment, the polymers and copolymers have a MW of up to about 450 kilodaltons, or up to about 600 kilodaltons, or up to about 1,000 kilodaltons.

Other examples of suitable polymers are commercially available from sources such as Sigma-Aldrich, and include, but are not limited to, polybutadiene, poly(butadiene-co-acrylonitrile), polybutadiene-block-polyisoprene, polybutadiene-graft-poly(methyl acrylate-co-acrylonitrile), polyisoprene, and partially hydrogenated polyisoprene.

Examples of suitable poly(ethylene-co-vinyl acetate) polymers include polymers having vinyl acetate concentrations of at least about 8%, or at least about 10%, at least about 20%, at least about 30%, and up to about 34%, up to about 40%, up to about 50%, or up to about 90%, in the form of beads, pellets, granules, etc. (commercially available are 12%, 14%, 18%, 25%, 33%). pEVA co-polymers with lower percent vinyl acetate become increasingly insoluble in typical solvents, whereas those with higher percent vinyl acetate become decreasingly durable.

One suitable polymer mixture includes mixtures of poly(butylmethacrylate) (pBMA) and poly(ethylene-co-vinyl acetate) co-polymers (pEVA). In one embodiment, the mixture includes absolute polymer concentrations (i.e., the total combined concentrations of both polymers in the coating composition), of at least about 0.25% (by weight) and up to about 70% (by weight). In another embodiment, the mixture includes individual polymer concentrations in the coating solution of at least about 0.05% (by weight) and up to about 70% (by weight). In one embodiment the polymer mixture includes poly(n-butylmethacrylate) (pBMA) with a molecular weight of at least about 100 kilodaltons and up to about 900 kilodaltons and a pEVA copolymer with a vinyl acetate content of at least about 24% (by weight) and up to about 36% (by weight). In one embodiment the polymer mixture includes poly(n-butylmethacrylate) with a molecular weight of at least about 200 kilodaltons and up to about 400 kilodaltons and a pEVA copolymer with a vinyl acetate content of at least about 30% (by weight) and up to about 34% (by weight).

Optionally, the polymeric mixture may include one or more additional polymers in combination with the first and second polymer components. In one embodiment, the polymeric mixture includes one or more additional polymers selected from (i) poly(alkylene-co-alkyl(meth)acrylates, (ii) ethylene copolymers with other alkylenes, (iii) polybutenes, (iv) diolefin-derived, non-aromatic polymers and copolymers, (v) aromatic group-containing copolymers, (vi) epichlorohydrin-containing polymers, and (vii) poly(ethylene-co-vinyl acetate). Generally, if one or more additional polymers are included, the one or more additional polymers are different from the first or second polymer component used in the coating composition. In one embodiment, the additional polymers may be substituted for up to about 25% of the second polymer. In other embodiments, the additional polymers may be substituted for up to about 50% of the second polymer.

The mixtures of polymers can include absolute polymer concentrations (i.e., the total combined concentrations of both polymers in the coating composition), of at least about 0.1% (by weight), or at least about 5% (by weight), or at least about 15% by weight, or at least about 25% (by weight) and up to about 35% (by weight), or up to about 50% (by weight), or up to about 75% (by weight), or up to about 85% (by weight), or up to about 95% (by weight). Various polymer mixtures include at least about 10% (by weight) of either the first polymer or the second polymer.

In one embodiment, both first and second polymer components are purified for such use to a desired extent and/or provided in a form suitable for in vivo use. In other embodiments, biocompatible additives may be added, such as dyes and pigments (e.g., titanium dioxide, Solvent Red 24, iron oxide, and Ultramarine Blue); slip agents (e.g., amides such as oleyl palmitamide, N,N′-ethylene bisoleamide, erucamide, stearamide, and oleamide); antioxidants (e.g. butylated hydroxytoluene (BHT), vitamin E (tocopherol), BNX™, dilauryl thiodipropionate (DLTDP), IrganoX™ series, phenolic and hindered phenolic antioxidants, organophosphites (e.g., trisnonylphenyl phosphite, Irgafos™ 168), lactones (e.g., substituted benzofuranone), hydroxylamine, and MEHQ (monomethyl ether of hydroquinone)); surfactants (e.g., anionic fatty acid surfactants (e.g., sodium lauryl sulfate, sodium dodecylbenzenesulfonate, sodium stearate, and sodium palmitate), cationic fatty acid surfactants (e.g., quaternary ammonium salts and amine salts), and nonionic ethoxylated surfactants (e.g., ethoxylated p-octylphenol)); and leachable materials (i.e., permeation enhancers) (e.g., hydrophilic polymers (e.g., poly(ethylene glycol), polyvinylpyrrolidone, and poly(vinyl alcohol)) and hydrophilic small molecules (e.g., sodium chloride, glucose)). In addition, any impurities may be removed by conventional methods available to those skilled in the art.

### Conductive Material

To impart conductivity to the coating composition, the composition can include an electrically conductive material combined with the polymeric mixture. As used herein, the term “electrically conductive material” refers to a material that is capable of conducting an electric current. In one embodiment, an “electrically conductive material” includes inorganic conductive fillers, such as carbon nanotubes. For carbon nanotubes, the resistivity may be in the range 1E-3 to 1E-5 ohm*cm. In another embodiment, the electrically conductive material can include one or more organic conductive polymers. For example, an electrically conducting formulation of polypyrrole or PEDOT may have a resistivity in the range 1E-2 to 1E-4 ohm*cm.

In one embodiment, the electrically conductive material is dispersed throughout the polymer mixture. As used herein, the term “dispersed” means that the electrically conductive material is distributed more or less evenly throughout the polymeric mixture. In one embodiment, the dispersion is a suspension. In a suspension, particles or domains of the electrically conductive material are dispersed more or less evenly throughout the nonconductive polymer matrix. The relative amount of the electrically conductive material within the polymeric mixture can be adjusted to alter the electrical properties of coating composition, such as impedance, conductivity, and/or resistance. In general, a sufficient amount of electrically conductive material is included to allow for electron transport or conduction within and across the polymeric matrix. In one embodiment, the conductive polymeric coating composition includes at least about 0.5% (by weight), at least about 10% (by weight), or at least about 20% (by weight) and up to about 40% (by weight), or up to about 50% (by weight) of an electrically conductive material. In another embodiment, the coating composition includes domains or discrete regions of electrically conductive material interspersed within the polymer matrix.

As used herein, the term “polymer” refers to a class of natural or synthetic macromolecules composed of monomeric subunits. While in some polymers the monomeric subunits may all be the same, the monomeric subunits need not all be the same or have the same structure. Polymers can include long chains of unbranched or branched monomers and can include cross-linked networks of monomers in two or three dimensions.

As used here, the term “conductive polymers” refers to polymers that are capable of conducting electricity. In traditional non-conducting polymers, the valence electrons are bound in sp^{a} hybridized covalent bonds. Such “sigma-bonding electrons” have low mobility and do not contribute to the electrical conductivity of the material. In contrast, conducting polymers generally have a backbone of contiguous sp² hybridized carbon centers. One valence electron on each center resides in a p_{z} orbital, which is orthogonal to the other three sigma-bonds. The electrons in these delocalized orbitals have high mobility, when the material is “doped” by oxidation, which removes some of these delocalized electrons. Thus the p-orbitals form a band, and the electrons within this band become mobile when it is partially emptied. In principle, these same materials can be doped by reduction, which adds electrons to an otherwise unfilled band. In practice, most conductive polymers are doped oxidatively to give p-type materials.

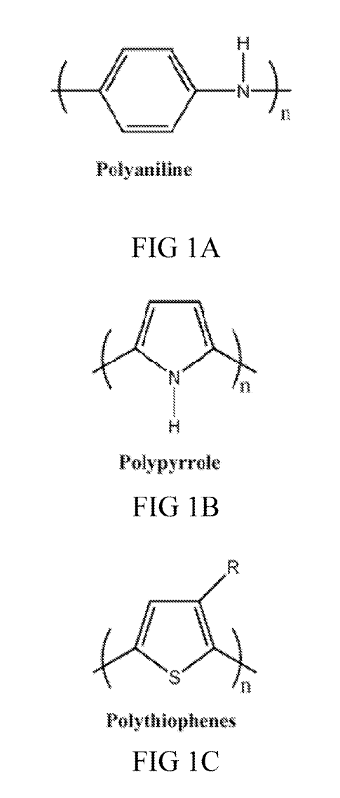

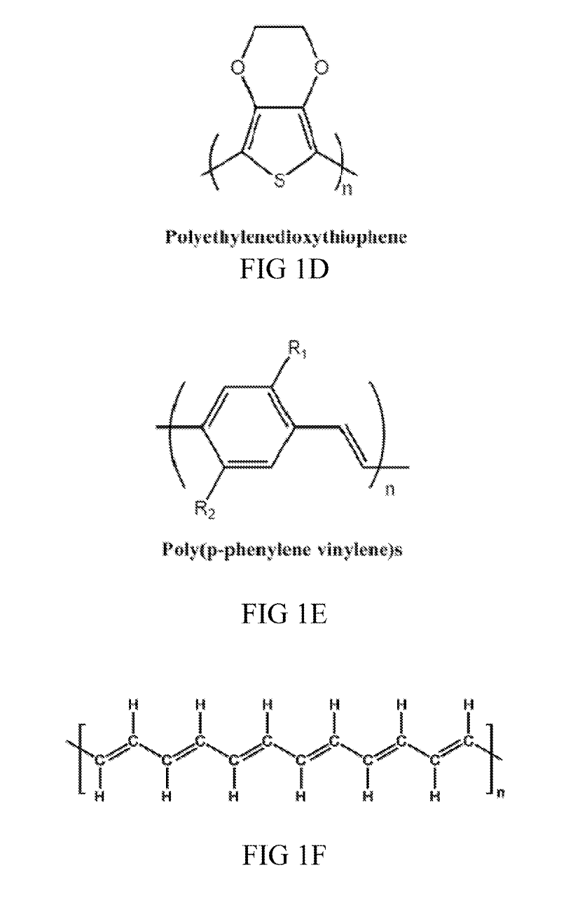

As used herein, the term “organic conductive polymers” refers to polymers that have carbon molecules in the polymer backbone. In one embodiment, the organic conductive polymer has alternating single and double bonds along the polymer backbone. Well-studied classes of organic conductive polymers include poly(acetylene), poly(pyrrole), poly(thiophene), poly(aniline), poly(arylene), poly(p-phenylene sulfide), and poly(para-phenylene vinylene). Other less well studied conductive polymers include polyindole, polypyrene, polycarbazole, polyazulene, polyazepine, polyfluorene, and polynaphthalene. Specific examples of organic conductive polymers include poly(3,4-ethylenedioxythiophene) (PEDOT), poly(phenylene vinylene) (PPV), polyspirobifluorene, poly(3-hexylthiophene), poly(o-methoxyaniline) (POMA), and poly(ophenylenediamine) (PPD). Examples of monomeric subunits for various conductive polymers are shown schematically in FIGS. 1A-F. FIG. 1A: polyaniline; FIG. 1B: polypyrrole; FIG. 1C: polythiophene; FIG. 1D: polyethylenedioxythiophene; FIG. 1E: poly(p-phenylene vinylene); and FIG. 1F: a conductive polymer with alternating single and double bonds along the carbon backbone.

In an alternate embodiment, the electrically conductive material includes inorganic conductive fillers. As used herein, the term “inorganic conductive filler” refers to inorganic materials that are electrically conductive and include metals, oxides and ceramic precursors, and carbon allotropes. Examples of suitable metals include such as metal micro or nanoparticles, including but not limited to silver, gold, nickel, copper, iron oxide, tin, and mixtures and combinations thereof. Metal coated beads or microparticles can also be used. Suitable oxides and ceramic precursors include, but are not limited to, silicon oxide, aluminum oxide, boron nitride, and aluminum nitride. Examples of suitable carbon allotropes include diamond, fullerenes such as carbon nanotubes, carbon nanowires, or carbon naonospheres, and carbon black. In one embodiment, the electrically conductive inorganic filler is dispersed within the conductive coating composition. The amount of inorganic conductive filler included in the coating composition can vary, depending on the need to have sufficient conductive material for the formation of conductive “bridges” within the polymeric matrix and balanced on the other hand with potential adverse impact on the mechanical properties of the coating composition

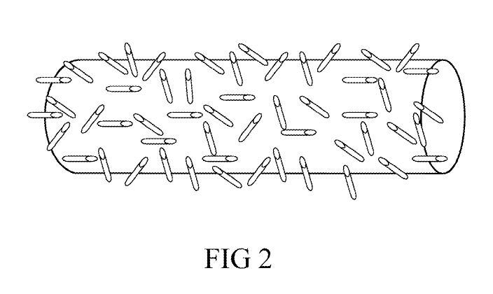

Carbon nanotubes (CNTs) are cylindrical members of the fullerene structural family, which also includes spherical “buckyballs.” The diameter of a nanotube is generally between about 1 and about 5 nanometers, more typically between about 1 and about 2 nanometers. The length of the nanotube can extend from a few nanometers (at least about 1, or at least about 10 nanometers), up to several millimeters in length. Nanotubes can include single walled nanotubes (SWNTs) and multi-walled nanotubes (MWNTs). Multi-walled nanotubes (MWNT) are made of multiple rolled layers (concentric tubes) of graphite. Carbon nanotubes are excellent conductors of electricity. In one embodiment, the coating composition includes carbon nanotubes protruding from the coating surface, as shown in FIG. 2. While not wishing to be bound by theory, it is believed that the presence of the nanotube protrusions can increase the surface area of the device, in addition to increasing conductivity.

Carbon black is a form of amorphous carbon that has a high surface area to volume ratio. In general, carbon black is a material produced by the incomplete combustion of heavy petroleum products. Carbon black can be used to impart electrical conductivity to plastics by the formation of “bridges” the conductive additives when a sufficient amount of carbon black included in the polymeric matrix. In general, as the loading of the carbon black in the polymeric composition increases, the polymeric matrix remains insulating, until the percolation threshold is met, in which the conductivity of the composition passes through a sharp and abrupt rise over a very narrow black concentration (loading) range. Further increases in loading past this threshold tend to cause little increase in conductivity but can adversely impact the mechanical properties of the coating composition.

### Bioactive Agent

In one embodiment, the coating composition includes one or more bioactive agents. The terms “bioactive agent,” “biologically active agent” and “active agent” are used interchangeably herein to refer to a wide range of biologically active materials or drugs that can be incorporated into the coating composition. In one embodiment, more than one active agent can be used. In another embodiment, the coating composition includes co-agents or co-drugs which may act differently than the first agent or drug and may have an elution profile that is different than the first agent or drug.

In one embodiment, the bioactive agents can be included in one or more layers or coatings. In one embodiment, the bioactive agent is included in a pretreatment layer. In another embodiment, the bioactive agent is included in a protective coating or topcoat. In one embodiment, the bioactive agent in the coating composition may be the same as or different than the bioactive agent included in the pretreatment coating and/or protective coating or topcoat. Further, such bioactive agents may sometimes be referred to herein as the “pretreatment coating bioactive agent” or the “protective coating bioactive agent” or “topcoat bioactive agent.”

The various ingredients and relative amounts thereof in the composition can be adjusted to alter the release kinetics for any particular bioactive agent. While not intending to be bound by theory, the release kinetics of the bioactive agent in vivo are thought to generally include both a short term (“burst”) release component, within the order of minutes to hours after implantation, and a longer term release component, which can range from on the order of hours to days or even months or years of useful release. In one embodiment, the amount and rate of release of agent(s) from the medical device can be controlled by adjusting the relative types and/or concentrations of hydrophobic polymers in the mixture. For a given combination of polymers, for instance, this approach permits the release rate to be adjusted and controlled by simply adjusting the relative concentrations of the polymers in the coating mixture. This provides an additional means to control rate of bioactive agent release besides the conventional approach of varying the concentration of bioactive agent in a coated composition.

In one embodiment, the bioactive agent(s) do not chemically interact with the coating composition during fabrication or during the bioactive agent release process. In another embodiment, the active agent can be in a microparticle. In one embodiment, the microparticles can be dispersed on the surface of the substrate.

In one embodiment, the bioactive agents are dispersed throughout the coating composition or matrix. In one embodiment, the bioactive agent forms a true solution with the solvent. In another embodiment, the bioactive agent is included as a suspension within the coating composition. In another embodiment, one or more bioactive agents are encapsulated within an inner matrix structure, for example, a microparticle structure formed of semipermeable cells and/or degradable polymers. One or more inner matrices may be placed in one or more locations within the coating composition and at one or more locations in relation to the substrate. Examples of inner matrices, for example degradable encapsulating matrices formed of semipermeable cells and/or degradable polymers, are disclosed and/or suggested in U.S. Publication No. 20030129130, U.S. Patent Application Ser. No. 60/570,334 filed May 12, 2004, U.S. Patent Application Ser. No. 60/603,707, filed Aug. 23, 2004, U.S. Publication No. 20040203075, filed Apr. 10, 2003, U.S. Publication No. 20040202774 filed on Apr. 10, 2003, and U.S. patent application Ser. No. 10/723,505, filed Nov. 26, 2003, the entire contents of which are incorporated by reference herein.

The biologically active agent can be applied to the device to provide a therapeutically effective amount of the agent to a patient receiving the coated device. The weight of the coating attributable to the active agent can be in any range desired for a given active agent in a given application. In some embodiments, weight of the coating attributable to the active agent is in the range of about 1 microgram to about 10 milligrams of active agent per cm² of the effective surface area of the device. By “effective” surface area it is meant the surface amenable to being coated with the composition itself. For a flat, nonporous, surface, for instance, this will generally be the macroscopic surface area itself, while for considerably more porous or convoluted (e.g., corrugated, pleated, or fibrous) surfaces the effective surface area can be significantly greater than the corresponding macroscopic surface area. In one embodiment, the weight of the coating attributable to the active agent is at least about 0.01 mg and up to about 0.5 mg of active agent per cm² of the gross surface area of the device.

The concentration of the bioactive agent or agents dissolved or suspended in the coating can range from at least about 0.01% (by weight) and up to about 50% (by weight), or up to about 90% (by weight), based on the weight of the final coating composition. In one embodiment, the bioactive agent is included in an amount of at least about 1% (by weight), or at least about 5% (by weight), or at least about 25% (by weight), and up to about 45% (by weight), or up to about 60% (by weight), or up to about 75% of a mixture that includes the first polymer, second polymer, and bioactive agent (i.e., excluding solvents and other additives).

In one embodiment, the active agent is hydrophilic. In one embodiment, the active agent has a molecular weight of less than 1500 daltons and a water solubility of greater than 10 mg/mL at 25° C. In other embodiments, the active agent is hydrophobic. In one embodiment, the active agent can have a water solubility of less than 10 mg/mL at 25° C.

Suitable bioactive (e.g., pharmaceutical) agents include virtually any therapeutic substance which possesses desirable therapeutic characteristics for application to the implant site. A comprehensive listing of bioactive agents can be found in The Merck Index, Thirteenth Edition, Merck & Co. (2001), the entire contents of which is incorporated by reference herein. In one embodiment, the bioactive agent includes an anti-inflammatory agent such as a corticosteroid. In another embodiment, the bioactive agent includes an anti-proliferative, anti-biotice, or anti-microbial agent. Bioactive agents are commercially available from Sigma Aldrich (e.g., vincristine sulfate). Additives such as inorganic salts, BSA (bovine serum albumin), and inert organic compounds can be used to alter the profile of bioactive agent release, as known to those skilled in the art.

Active agents can include many types of therapeutics including thrombin inhibitors, antithrombogenic agents, thrombolytic agents, fibrinolytic agents, anticoagulants, anti-platelet agents, vasospasm inhibitors, calcium channel blockers, steroids, vasodilators, anti-hypertensive agents, antimicrobial agents, antibiotics, antibacterial agents, antiparasite and/or antiprotozoal solutes, antiseptics, antifungals, angiogenic agents, anti-angiogenic agents, inhibitors of surface glycoprotein receptors, antimitotics, microtubule inhibitors, antisecretory agents, actin inhibitors, remodeling inhibitors, antisense nucleotides, anti-metabolites, miotic agents, antiproliferatives, anticancer chemotherapeutic agents, anti-neoplastic agents, antipolymerases, antivirals, anti-AIDS substances, anti-inflammatory steroids or non-steroidal anti-inflammatory agents, analgesics, antipyretics, immunosuppressive agents, immunomodulators, growth hormone antagonists, growth factors, radiotherapeutic agents, peptides, proteins, enzymes, extracellular matrix components, ACE inhibitors, free radical scavengers, chelators, anti-oxidants, photodynamic therapy agents, gene therapy agents, anesthetics, immunotoxins, neurotoxins, opioids, dopamine agonists, hypnotics, antihistamines, tranquilizers, anticonvulsants, muscle relaxants and anti-Parkinson substances, antispasmodics and muscle contractants, anticholinergics, ophthalmic agents, antiglaucoma solutes, prostaglandin, antidepressants, antipsychotic substances, neurotransmitters, anti-emetics, imaging agents, specific targeting agents, and cell response modifiers.

Other biologically useful compounds that can also be included in the coating material include, but are not limited to, hormones, (3-blockers, anti-anginal agents, cardiac inotropic agents, corticosteroids, analgesics, anti-inflammatory agents, anti-arrhythmic agents, immunosuppressants, anti-bacterial agents, anti-hypertensive agents, antimalarials, anti-neoplastic agents, anti-protozoal agents, anti-thyroid agents, sedatives, hypnotics and neuroleptics, diuretics, anti-parkinsonian agents, gastro-intestinal agents, anti-viral agents, anti-diabetics, anti-epileptics, anti-fungal agents, histamine H-receptor antagonists, lipid regulating agents, muscle relaxants, nutritional agents such as vitamins and minerals, stimulants, nucleic acids, polypeptides, and vaccines.

Antibiotics are substances which inhibit the growth of or kill microorganisms. Antibiotics can be produced synthetically or by microorganisms. Examples of antibiotics include penicillin, tetracycline, chloramphenicol, minocycline, doxycycline, vancomycin, bacitracin, kanamycin, neomycin, gentamycin, erythromycin, geldanamycin, geldanamycin analogs, cephalosporins, or the like. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

Antiseptics are recognized as substances that prevent or arrest the growth or action of microorganisms, generally in a nonspecific fashion, e.g., either by inhibiting their activity or destroying them. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Antiviral agents are substances capable of destroying or suppressing the replication of viruses. Examples of anti-viral agents include a-methyl-ladamantanemethylamine, hydroxy-ethoxymethylguanine, adamantanamine, 5-iodo-2′-deoxyuridine, trifluorothymidine, interferon, and adenine arabinoside.

Enzyme inhibitors are substances that inhibit an enzymatic reaction. Examples of enzyme inhibitors include edrophonium chloride, N-methylphysostigmine, neostigmine bromide, physostigmine sulfate, tacrine HCL, tacrine, 1-hydroxy maleate, iodotubercidin, p-bromotetramisole, 10-(a-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylaminie, N-monomethyl-L-arginine acetate, carbidopa, 3-hydroxybenzylhydrazine HCl, hydralazine HCl, clorgyline HCl, deprenyl HCl L(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthne, papaverine HCl, indomethacind, 2-cyclooctyl-2-hydroxyethylamine hydrochloride, 2,3-dichloro-a-methylbenzylamine (DCMB), 8,9-dichloro-2,3,4,5-tetrahydro-1H-2-benzazepine hydrochloride, p-aminoglutethimide, p-aminoglutethimide tartrate R(+), paminoglutethimide tartrate S(−), 3-iodotyrosine, alpha-methyltyrosine L(−), alphamethyltyrosine D(−), cetazolamide, dichlorphenamide, 6-hydroxy-2-benzothiazolesulfonamide, and allopurinol.

Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide.

Local anesthetics are substances that have an anesthetic effect in a localized region. Examples of such anesthetics include procaine, lidocaine, tetracaine and dibucaine.

Imaging agents are agents capable of imaging a desired site, e.g., tumor, in vivo. Examples of imaging agents include substances having a label that is detectable in vivo, e.g., antibodies attached to fluorescent labels. The term antibody includes whole antibodies or fragments thereof.

Cell response modifiers are chemotactic factors such as platelet-derived growth factor (PDGF). Other chemotactic factors include neutrophil-activating protein, monocyte chemoattractant protein, macrophage-inflammatory protein, SIS (small inducible secreted), platelet factor, platelet basic protein, melanoma growth stimulating activity, epidermal growth factor, transforming growth factor alpha, fibroblast growth factor, platelet-derived endothelial cell growth factor, insulin-like growth factor, nerve growth factor, bone growth/cartilage-inducing factor (alpha and beta), and matrix metalloproteinase inhibitors. Other cell response modifiers are the interleukins, interleukin receptors, interleukin inhibitors, interferons, including alpha, beta, and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins, antisense molecules, androgenic receptor blockers and statin agents.

In other embodiments the active agent can include heparin, covalent heparin, synthetic heparin salts, or another thrombin inhibitor; hirudin, hirulog, argatroban, D-phenylalanyl-L-poly-L-arginyl chloromethyl ketone, or another antithrombogenic agent; urokinase, streptokinase, a tissue plasminogen activator, or another thrombolytic agent; a fibrinolytic agent; a vasospasm inhibitor; a calcium channel blocker, a nitrate, nitric oxide, a nitric oxide promoter, nitric oxide donors, dipyridamole, or another vasodilator; HYTRIN™ or other antihypertensive agents; a glycoprotein IIb/IIIa inhibitor (abciximab) or another inhibitor of surface glycoprotein receptors; aspirin, ticlopidine, clopidogrel or another antiplatelet agent; colchicine or another antimitotic, or another microtubule inhibitor; dimethyl sulfoxide (DMSO), a retinoid, or another antisecretory agent; cytochalasin or another actin inhibitor; cell cycle inhibitors; remodeling inhibitors; deoxyribonucleic acid, an antisense nucleotide, or another agent for molecular genetic intervention; methotrexate, or another antimetabolite or antiproliferative agent; tamoxifen citrate, TAXOL®, paclitaxel, or the derivatives thereof, rapamycin (or other rapalogs), vinblastine, vincristine, vinorelbine, etoposide, tenopiside, dactinomycin (actinomycin D), daunorubicin, doxorubicin, idarubicin, anthracyclines, mitoxantrone, bleomycin, plicamycin (mithramycin), mitomycin, mechlorethamine, cyclophosphamide and its analogs, chlorambucil, ethylenimines, methylmelamines, alkyl sulfonates (e.g., busulfan), nitrosoureas (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, morpholino phosphorodiamidate oligomer or other anti-cancer chemotherapeutic agents; cyclosporin, tacrolimus (FK-506), pimecrolimus, azathioprine, mycophenolate mofetil, mTOR inhibitors, or another immunosuppressive agent; cortisol, cortisone, dexamethasone, dexamethasone sodium phosphate, dexamethasone acetate, dexamethasone derivatives, betamethasone, fludrocortisone, prednisone, prednisolone, 6U-methylprednisolone, triamcinolone (e.g., triamcinolone acetonide), or another steroidal agent; trapidil (a PDGF antagonist), angiopeptin (a growth hormone antagonist), angiogenin, a growth factor (such as vascular endothelial growth factor (VEGF)), or an anti-growth factor antibody (e.g., ranibizumab, which is sold under the tradename LUCENTIS®), or another growth factor antagonist or agonist; dopamine, bromocriptine mesylate, pergolide mesylate, or another dopamine agonist; ⁶Co (5.3 year half life), ¹⁹²Ir (73.8 days), ³²P (14.3 days), ¹¹¹In (68 hours), ⁹⁰Y (64 hours), ⁹⁹Tc (6 hours), or another radiotherapeutic agent; iodine-containing compounds, barium-containing compounds, gold, tantalum, platinum, tungsten or another heavy metal functioning as a radiopaque agent; a peptide, a protein, an extracellular matrix component, a cellular component or another biologic agent; captopril, enalapril or another angiotensin converting enzyme (ACE) inhibitor; angiotensin receptor blockers; enzyme inhibitors (including growth factor signal transduction kinase inhibitors); ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³H-, ³²P- or ³⁶S-radiolabelled form or other radiolabelled form of any of the foregoing; an estrogen (such as estradiol, estriol, estrone, and the like) or another sex hormone; AZT or other antipolymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluorozinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin, or other antibody targeted therapy agents; gene therapy agents; enalapril and other prodrugs; PROSCAR®, HYTRIN® or other agents for treating benign prostatic hyperplasia (BHP); mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of any of these.

### Medical Device

The coating composition described herein is suitable for application to a medical device. Although the discussion herein emphasizes the benefits of the coating composition when used in connection with an implanted medical device, it is envisioned that the coating composition can also be used in connection with external medical devices that include one or more electrically conductive elements, such as stimulating or sensing electrodes. In one embodiment, the coating composition is applied to one or more electrically conductive elements of an implantable medical device. In one embodiment, the conductive polymer coating is used to coat at least part of one or more electrically conductive surfaces. As used herein, the term “electrically conductive surface” refers to a surface that is able to conduct an electric current. In one embodiment, the conductive polymer coating is used to coat the surface of an electrode of a medical device. As used herein, the term “electrode” can refer to both sensing and stimulating electrodes. Suitable electrodes can include any combination of one or more coil electrodes, tip electrodes, ring electrodes, multi-element coils, spiral coils, spiral coils mounted on non-conductive backing, and screen patch electrodes, for example. Suitable electrodes can be constructed using any conductive material. In one embodiment, one or more electrodes are constructed from a conductive metal material. In one embodiment, the conductive metal material is selected from: titanium, copper, stainless steel, gold, silver, platinum, platinum-iridium alloy, cobalt-chrome alloys, etc. In use, the electrode is typically located in close proximity or in direct contact with the appropriate tissue of the patient.

Sensing electrodes include electrodes that can detect electrical signals in tissue that are created by chemical reactions. Stimulating electrodes include electrodes configured to administer an electric impulse to a tissue of a patient. A variety of stimulating and sensing electrodes are known and used in connection with a variety of tissues and therapies. Examples of stimulating electrodes include cardiostimulators, neurostimulators, such as vagus nerve stimulators, carotid artery stimulators, cochlear implants, spinal stimulators, and gastric stimulators.

Well known medical devices with electrodes include those used in connection with pacemakers and defibrillators. However, other therapies that use electrodes include electrocardiography (ECG), electroencephalography (EEG), electromyography (EMG), electroretinography (ERG), electrosurgical devices, nasopharyngeal devices, pH electrodes, neurological devices, blood gas analyzers, and transcutaneous electrode simulation (TENS).

Approved uses for electrostimulation therapy include deep brain stimulation (DBS) or cortical therapy, used to treat essential tremor in Parkinson's disease and dystonia; cochlear stimulation to treat deafness; vagus nerve stimulation (VNS) therapy for the treatment of depression and epilepsy; peripheral nerve stimulation (PNS) for the treatment of chronic pain; spinal cord stimulation (SCS) for the treatment of chronic pain and angina; spinal stimulation for the treatment of chronic pain, malignant pain and spasticity; pulmonary stimulation for respiratory support; sacral nerve stimulation (SNS) for the treatment of pelvic or urinary pain, as well as incontinence. Other uses include: DBS/cortical stimulation for the treatment of obsessive-compulsive disorder, depression, tinnitus, epilepsy, stroke, pain, coma, paralysis, Tourette's, memory loss, gait and eating disorders; brain stimulation for the treatment of epilepsy, Parkinson's, and Alzheimer's; the use of an artificial retina for the treatment of retinitis pigmentosa; occipital nerve stimulation (ONS) for the treatment of headaches and migraines; vagal nerve stimulation (VNS) for the treatment of congestive heart failure or obesity; spinal cord stimulation for the treatment of peripheral vascular disease (PVD) pain or diabetic peripheral neuropathy (DPN); spinal stimulation for the treatment of amyotrophic lateral sclerosis (ALS) or Huntington's; gastric stimulation for the treatment of obesity, gastroparaesis, or irritable bowel syndrome; and sacral nerve stimulation for the treatment of pelvic pain or sexual dysfunction.

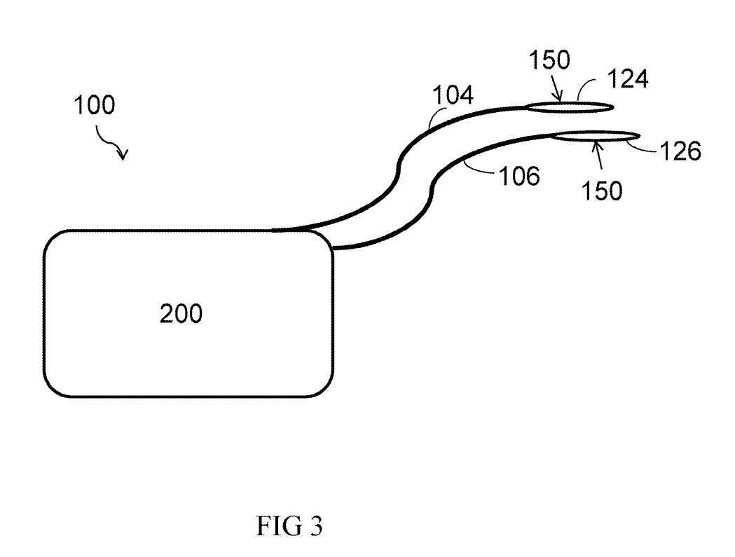

FIG. 3 is a schematic view of an implantable medical device 100 with an electrically conductive element 110. In the embodiment shown in FIG. 1, the device 100 includes a housing 200 that encases the electronics for the device 100, one or more leads 104, 106 that electrically couple the electronics located within the housing 200, to one or more electrodes 124, 134 that are disposed in operative relation to the patient's tissue. The electrodes 124, 134 can include a coating composition 150 on at least a part of the electrically conductive surface. In one embodiment, the housing includes a pulse generator 102 that can generate pulses and/or therapeutic shocks which are delivered through the leads 104, 106 and electrodes 124, 134 to the tissue of the patient.

### Method

In one embodiment, the coating composition is used to coat the surface of one or more electrically conductive elements of a medical device. Advantageously, the coating composition described herein adheres well to conductive metal surfaces.

In one embodiment, a composition is prepared that includes one or more solvents, a combination of polymers dissolved in the solvent(s) and an electrically conductive material. In one embodiment, the electrically conductive material is dispersed throughout the polymeric mixture. In another embodiment, discrete domains or clusters of electrically conductive material is interspersed within the polymeric mixture. In yet another embodiment, the coating composition includes one or more bioactive agent or agents dissolved or suspended in the mixture.

The coating composition can be provided in any suitable form, e.g., in the form of a true solution, or fluid or paste-like emulsion, mixture, dispersion or blend. In one embodiment, the solvent is one in which the polymers of the polymeric mixture form a true solution. The electrically conductive material and/or bioactive agent may either be soluble in the solvent or form a suspension or dispersion throughout the solvent. In one embodiment, one or more solvents are not only capable of dissolving the polymers in solution, but are sufficiently volatile to permit the composition to be effectively applied to a surface (e.g., by spraying) and quickly removed (e.g., by drying) to provide a stable and desirable coated composition. In one embodiment, the coated composition is homogeneous, with the first and second polymers effectively serving as cosolvents for each other, with the electrically conductive material and/or bioactive agent substantially equally sequestered within them both.

Suitable solvents include, but are not limited to, alcohols (e.g., methanol, butanol, propanol and isopropanol), alkanes (e.g., halogenated or unhalogenated alkanes such as hexane, cyclohexane, methylene chloride and chloroform), amides (e.g., dimethylformamide), ethers (e.g., tetrahydrofuran (THF), dioxolane, and dioxane), ketones (e.g., methyl ethyl ketone), aromatic compounds (e.g., toluene and xylene), nitriles (e.g., acetonitrile) and esters (e.g., ethyl acetate). In some embodiments, THF and chloroform have been found to be effective solvents due to their excellent solvency for a variety of polymers and bioactive agents.

The resultant composition can be applied to the device in any suitable fashion, e.g., it can be applied directly to the surface of the medical device, or alternatively, to the surface of a surface-modified medical device, by dipping, spraying, or any conventional technique. The method of applying the coating composition to the device is typically governed by the geometry of the device and other process considerations. The coating is cured by evaporation of the solvent. The curing process can be performed at room temperature, elevated temperature, or with the assistance of vacuum. The applied coating composition is cured (e.g., by solvent evaporation) to provide a tenacious and flexible composition on the surface of the medical device.

In some embodiments it may be desirable to increase the coating surface area. For example the coating composition can be applied in such a way to provide a porous coating. In another embodiment, the conductive coating additive (such as carbon nanotubes) may partially protrude from the coating to increase the surface area.

The overall weight of the coating upon the surface can vary. In general, the overall weight of is determined by the desired function of the coating. In one embodiment, the desired weight of the coating is guided by the quantity of drug required to provide adequate activity under physiological conditions. In another embodiment, the desired weight of the coating is guided by the desired impedance, surface area or roughness of the device. Similarly, the thickness of the coating composition can vary. In one embodiment, the thickness of the coating composition is at least about 1 micrometer, or at least about 5 micrometers, and up to about 100 micrometers, or up to about 200 micrometers.

In one embodiment, the coating is applied to a device under conditions of controlled relative humidity (at a given temperature), for instance, under conditions of increased or decreased relative humidity as compared to ambient humidity. Humidity can be “controlled” in any suitable manner, including at the time of preparing and/or using (as by applying) the composition, for instance, by coating the surface in a confined chamber or area adapted to provide a relative humidity different than ambient conditions, and/or by adjusting the water content of the coating or coated composition itself. Without intending to be bound by theory, it appears that the elution rate of a bioactive agent from a coating composition generally increases as relative humidity increases.

In one embodiment, the surface of a medical device may be roughened to increase adhesion of the coating composition to the medical device and/or alter elution profiles. Without intending to be bound by theory, it is believed that roughening of the surface provides for a greater surface area between the coating composition and the surface of the medical device, which may increase adhesion. Further, in embodiments with relatively aggressive roughening and/or relatively thin coatings, the peaks and valleys of the roughened surface may transfer through the coating composition, thereby increasing the surface area of the coating. Such increased surface area may alter the bioactive agent release profile in situ.

The surface of the medical device may be roughened by any suitable method. In one embodiment, the surface of the medical device may be roughened by projecting silica particles at the surface. The extent of the roughening may be characterized by peak to valley distances. For example, the extent of roughening may be characterized by the distance between the average of the ten highest peaks and the ten lowest valleys. In one embodiment, the extent of roughening may range from at least about 2 μm, or at least about 5 μm, or at least about 6.5 μm, and up to about 12 μm, or up to about 15 μm, or up to about 20 μm.

The coating composition can be applied using a plurality of individual steps or layers, in which the identity and/or relative amounts of the elements of the coating composition can be varied, including for example, the first and/or second polymer, the electrically conductive material, and the bioactive agent.

In one embodiment, the topcoat is hydrophilic, such as a photolinked hydrogel, and is electrically conductive. In another embodiment, the topcoat layer includes an electrically conductive material as described herein. In one embodiment, the topcoat layer includes a bioactive agent, which can be the same or different than the bioactive agent included in the polymeric coating. In another embodiment, the topcoat does not include a bioactive agent.

In one embodiment, one or more additional layers are applied on top of a coating layer that includes bioactive agent. Such additional layer(s) or “topcoats” can provide a number of benefits, such as biocompatibility enhancement, delamination protection, durability enhancement, and bioactive agent release control, to just mention a few. In one embodiment the topcoat may include one or more of the first, second, and/or additional polymers described herein without the inclusion of a bioactive agent. In one embodiment, the first or second polymers include functional groups (e.g. hydroxy, thiol, methylol, amino, and amine-reactive functional groups such as isocyanates, thioisocyanates, carboxylic acids, acyl halides, epoxides, aldehydes, alkyl halides, and sulfonate esters such as mesylate, tosylate, and tresylate) that can be used to bind the topcoat to the adjacent coating composition. Additionally, biocompatible topcoats (e.g. heparin, collagen, extracellular matrices, cell receptors . . . ) may be applied to the coating composition. Such biocompatible topcoats may be adjoined to the coating composition using known photochemical or thermochemical techniques. Additionally, release layers may be applied to the coating composition as a friction barrier layer or a layer to protect against delamination. Examples of biocompatible topcoats that may be used include those disclosed in U.S. Pat. Nos. 4,979,959 and 5,744,515.

In one embodiment, a hydrophilic topcoat may be provided. Such topcoats may provide several advantages, including providing a relatively more lubricious surface to aid in medical device placement in situ, as well as to further increase biocompatibility in some applications. Examples of hydrophilic agents that may be suitable for a topcoat includes polyacrylamide(36%)co-methacrylic acid(MA)-(10%)co-methoxy PEG1000MA-(4%)co-BBA-APMA compounds such as those described in example 4 of US Patent Application Publication No. 2002/0041899, photoheparin such as described in example 4 of U.S. Pat. No. 5,563,056, and a photoderivatized coating as described in Example 1 of U.S. Pat. No. 6,706,408, the contents of each of which is hereby incorporated by reference.

In some embodiments, the topcoat may be used to control the elution rate of a bioactive agent from a medical device surface. For example, topcoats may be described as the weight of the topcoat relative to the weight of the underlying bioactive agent containing layer. For example, the topcoat may be about 1 percent to about 50 percent by weight relative to the underlying layer. In some embodiments, the topcoat may be about 2 percent to about 25 percent by weight relative to the underlying layer. Optionally, in some embodiments, the topcoat may be about 5 percent to about 12 percent by weight relative to the underlying layer.

Applicants have found that providing a relatively thin topcoat compared to the underlying layer may significantly reduce initial drug elution rates to provide for longer elution times. For example, providing a topcoat weighing about 5% of the underlying layer may reduce initial elution rates (e.g., less than 20 hours) by more than about 50%.

In some embodiments, the topcoat layer includes a polymer that is also included in the underlying layer (e.g., first, second, and/or additional polymers as described above). Such topcoats may provide for superior adhesion between the top coat and the underlying layer.

Further, in some embodiments, one or more bioactive agents may be provided in a topcoat (sometimes referred to herein as a topcoat bioactive agent). The topcoat bioactive agent may be the same as or distinguishable from the bioactive agent included in an underlying layer. Providing bioactive agent within the topcoat allows for the bioactive agent to be in contact with surrounding tissue in situ while providing a longer release profile compared to coating compositions provided without topcoats. Such topcoats may also be used to further control the elution rate of a bioactive agent from a medical device surface, such as by varying the amount of bioactive agent in the topcoat. The degree to which the bioactive agent containing topcoat affects elution will depend on the specific bioactive agent within the topcoat as well as the concentration of the bioactive agent within the topcoat.

Any suitable amount of a bioactive agent may be included in the topcoat. For example, the upper limit of the amount of bioactive agent in the topcoat may be limited only by the ability of the topcoat to hold additional bioactive agent. In some embodiments, the bioactive agent may include about 1 to about 75 percent of the topcoat. Optionally, the bioactive agent may include about 5 to about 50 percent of the topcoat. In yet other embodiments, the bioactive agent may include about 10 to about 40 percent of the topcoat.

### WORKING EXAMPLES

### Example 1

A first coating composition was prepared by combining 30 mg/mL of a polymeric mixture containing poly(butyl(meth)acrylate)(“pBMA”), poly(ethylene-co-vinyl acetate) (“pEVA”) and polyaniline at a ratio of 1:1:1 (noted as polyaniline/pBMA/pEVA 1/1/1) in a solvent that included chloroform (CHCl₃) and xylene at a ratio of 2:1.

A second coating composition was prepared by combining 30 mg/mL of a polymeric mixture containing poly(butyl(meth)acrylate)(“pBMA”) and poly(ethylene-co-vinyl acetate) (“pEVA”) at a ratio of 1:1 (noted as pBMA/pEVA 1/1) in a solvent that included chloroform (CHCl₃) and xylene at a ratio of 2:1.

The coating composition were each applied to an I-vation™ intravitreal implant coil (SurModics, Eden Prairie, Minn.) using an Sonotek ultrasonic spray coater at ambient temperature.

The resistance of the two coatings, and an uncoated control I-vation coil was determined by submerging a stainless steel (SS) electrode in phosphate buffered saline (PBS) at a temperature of 21° C. along with the coated or uncoated I-vation coil at a distance of approximately 3 cm. A Fluke multimeter was attached to the stainless steel electrode and the cap of the I-vation coil to measure the resistance. The results are shown in the table below.

| | Average |
|---|---|
| Sample | Resistance |
| Uncoated (n = 4) | 5.18 M Ω |
| Bravo Only Coating (n = 5) | >100 M Ω |
| Polyaniline/Bravo (n = 5) | 7.25 M Ω |

Conclusion: The results clearly demonstrate that the inclusion of an electrically conductive material within a polymeric coating composition can substantially decrease the resistance of the material.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. It should be readily apparent that any one or more of the design features described herein may be used in any combination with any particular configuration. With use of the metal injection molding process, such design features can be incorporated without substantial additional manufacturing costs. That the number of combinations are too numerous to describe, and the present invention is not limited by or to any particular illustrative combination described herein. The scope of the present subject matter should be determined with reference to the appended claims, along with the full scope of equivalents to which such claims are entitled.

What is claimed is:

1. An electrically conductive coating composition comprising:
(a) a polymeric mixture comprising a plurality of polymers, including a first polymer component selected from the group consisting of: poly(alkyl)(meth)acrylates and poly(aromatic(meth)acrylates); and a second polymer component selected from the group consisting of: ethylene copolymers with other alkylenes; polybutenes; aromatic group-containing copolymers; epichlorohydrin-containing polymers; poly(alkylene-co-alkyl(meth)acrylates); diolefin-derived, non-aromatic polymers and copolymers; poly(ethylene-co-vinyl acetate), and combinations and mixtures thereof; and
(b) an electrically conductive material combined with the polymeric mixture.

2. The electrically conductive coating composition of claim 1, further comprising one or more bioactive agents dispersed within the polymeric mixture.

3. The electrically conductive coating composition of claim 1, wherein the electrically conductive material comprises a conductive polymeric material.

4. The electrically conductive coating composition of claim 3, wherein the conductive polymeric material is selected from the group consisting of poly(acetylene), poly(pyrrole), poly(thiophene), polyaniline, polythiophene, poly(p-phenylene sulfide), poly(para-phenylene vinylene), polyindole, polypyrene, polycarbazole, polyazulene, polyazepine, poly(fluorene), polynaphthalene, and combinations and mixtures thereof.

5. The electrically conductive coating composition of claim 3, wherein the conductive polymeric material is selected from the group consisting of polyaniline, polypyrrole, poly(3,4-ethylenedioxythiophene) (PEDOT), polyacetylene, Para-phenylene diamine (PPD), and combinations and mixtures thereof.

6. The electrically conductive coating composition of claim 1, wherein the electrically conductive material comprises biocompatible inorganic electrically conducting filler.

7. The electrically conductive coating composition of claim 6, wherein the inorganic electrically conducting filler is selected from the group consisting of: carbon nanotubes, metal micro/nano-particles, and carbon black.

8. The electrically conductive coating composition of claim 1 comprising at least about 0.5% by weight and up to about 50% by weight electrically conductive material.

9. The electrically conductive coating composition of claim 1, wherein the bioactive agent is selected from the group consisting of anti-inflammatory agents, anti-proliferative agents, antibiotics and antimicrobial agents.

10. A durable electrically conductive coating composition, comprising:
(a) a non-toxic hydrophobic polymeric mixture;
(b) an electrically conductive material dispersed throughout the polymeric mixture to form a polymeric coating having a resistivity and/or impedance sufficient to allow the electrical performance of the associated device; and
(c) one or more bioactive agents combined with the polymeric mixture.

11. The medical device of claim 10, wherein the electrically conductive material comprises a conductive polymeric material.

12. The medical device of claim 11, wherein the conductive polymeric material is selected from the group consisting of poly(acetylene), poly(pyrrole), poly(thiophene), polyaniline, polythiophene, poly(p-phenylene sulfide), poly(para-phenylene vinylene), polyindole, polypyrene, polycarbazole, polyazulene, polyazepine, poly(fluorene), polynaphthalene, and combinations and mixtures thereof.

13. The medical device of claim 10, wherein the electrically conductive material comprises biocompatible inorganic electrically conducting filler.

14. The medical device of claim 13, wherein the inorganic electrically conducting filler is selected from the group consisting of: carbon nanotubes, metal micro/nano-particles, and carbon black.

15. A medical device, comprising:
a. one or more electrically conductive elements having one or more surfaces;
b. a coating composition applied to at least part of one or more surfaces of the electrically conductive elements, wherein the coating composition comprises:
i. a polymeric mixture comprising a plurality of polymers, including a first polymer component selected from the group consisting of: poly(alkyl)(meth)acrylates and poly(aromatic(meth)acrylates); and a second polymer component selected from the group consisting of: ethylene copolymers with other alkylenes; polybutenes; aromatic group-containing copolymers; epichlorohydrin-containing polymers; poly(alkylene-co-alkyl(meth)acrylates); diolefin-derived, non-aromatic polymers and copolymers; poly(ethylene-co-vinyl acetate), and combinations and mixtures thereof; and
ii. an electrically conductive material and one or more bioactive agents combined with the polymeric mixture.

16. The medical device of claim 15, wherein one or more electrically conductive elements comprise a sensing or a stimulating electrode.

17. The medical device of claim 15, wherein the electrically conductive material comprises a conductive polymeric material.

18. The medical device of claim 17, wherein the conductive polymeric material is selected from the group consisting of poly(acetylene), poly(pyrrole), poly(thiophene), polyaniline, polythiophene, poly(p-phenylene sulfide), poly(para-phenylene vinylene), polyindole, polypyrene, polycarbazole, polyazulene, polyazepine, poly(fluorene), polynaphthalene, and combinations and mixtures thereof.

19. The medical device of claim 15, wherein the electrically conductive material comprises biocompatible inorganic electrically conducting filler.

20. The medical device of claim 19, wherein the inorganic electrically conducting filler is selected from the group consisting of: carbon nanotubes, metal micro/nano-particles, and carbon black.
